(19) United States
(12) Patent Application Publication
(10) Pub. No.: US 2024/0266052 A1
(43) Pub. Date: Aug. 8, 2024

(54) STRESSOR ESTIMATION DEVICE, STRESSOR ESTIMATION METHOD, AND STORAGE MEDIUM

(71) Applicant: NEC Corporation, Minato-ku, Tokyo (JP)

(72) Inventor: Tasuku KITADE, Tokyo (JP)

(73) Assignee: NEC Corporation, Minato-ku, Tokyo (JP)

(21) Appl. No.: 18/564,695

(22) PCT Filed: Jun. 1, 2021
(86) PCT No.: PCT/JP2021/020837

§ 371 Date: Nov. 28, 2023

(51) Int. Cl. G16H 50/20

(52) U.S. Cl. CPC G16H 50/20

(57) ABSTRACT

A stressor estimation device 1X mainly includes a chronic stress acquisition means 16X, a difference acquisition means 17X, and a stressor estimation means 18X. The chronic stress acquisition means 16X acquires a first chronic stress value of a target person in a first period and a second chronic stress value of the target person in a second period. The difference acquisition means 17X acquires difference information representing a difference between the first chronic stress value and the second chronic stress value and a difference between an event that occurred in the first period and an event that occurred in the second period. The stressor estimation means 18X estimates a stressor relating to a chronic stress of the target person, based on the difference information.

### TECHNICAL FIELD

The present disclosure relates to a technical field of a stressor estimation device, a stressor estimation method, and a storage medium for performing processing on estimation of a stressor.

### BACKGROUND

There is a device or a system for determining a stress state of a target person based on data measured from the subject. For example, Patent Literature 1 discloses a portable stress measuring device which determines a temporary stress degree of a target person on a daily basis based on examination data of the target person. Further, Patent Literature 2 discloses a stress estimation method of estimating a chronic stress based on schedule information.

### CITATION LIST

### Patent Literature

Patent Literature 1: JP 2007-275287A
Patent Literature 2: JP 2018-011721A

### SUMMARY

### Problem to be Solved

In general, since a part of the short term stress become chronic, it is an important factor in preventing and improving the chronic stress to identify the stressor that could lead to the chronic stress. On the other hand, Patent Literature 1 and Patent Literature 2 are silent on a specific technique for identifying a cause of the chronic stress.

In view of the above-described issues, it is an object of the present disclosure to provide a stressor estimation device, a stressor estimation method, and a storage medium capable of accurately estimating a stressor.

### Means for Solving the Problem

In one aspect of the stressor estimation device, there is provided a stressor estimation device including:
a chronic stress acquisition means configured to acquire a first chronic stress value of a target person in a first period and a second chronic stress value of the target person in a second period;
a difference acquisition means configured to acquire difference information representing a difference between the first chronic stress value and the second chronic stress value and a difference between an event that occurred in the first period and an event that occurred in the second period; and
a stressor estimation means configured to estimate a stressor relating to a chronic stress of the target person, based on the difference information.

In one aspect of the stressor estimation method, there is provided a stressor estimation method executed by a computer, the control method including:
acquiring a first chronic stress value of a target person in a first period and a second chronic stress value of the target person in a second period;
acquiring difference information representing a difference between the first chronic stress value and the second chronic stress value and a difference between an event that occurred in the first period and an event that occurred in the second period; and
estimating a stressor relating to a chronic stress of the target person, based on the difference information.

It is noted that the “computer” includes any electronic device (may be a processor included in the electronic device) and may be configured by a plurality of electronic devices.

In one aspect of the storage medium, there is provided a storage medium storing a program executed by a computer, the program causing the computer to:
acquire a first chronic stress value of a target person in a first period and a second chronic stress value of the target person in a second period;
acquire difference information representing a difference between the first chronic stress value and the second chronic stress value and a difference between an event that occurred in the first period and an event that occurred in the second period; and
estimate a stressor relating to a chronic stress of the target person, based on the difference information.

### Effect

An example advantage according to the present invention is to accurately estimate a stressor of a target person.

### BRIEF DESCRIPTION OF THE DRAWINGS

FIG. 1 It illustrates a schematic configuration of a stress management system according to a first example embodiment.

FIG. 2 It illustrates an example of a hardware configuration of a stressor estimation device common to each example embodiment.

FIG. 3 It is an example of a function block diagram of the stressor estimation device according to the first example embodiment.

FIG. 4 It is a diagram showing a specific example of generating difference information.

FIG. 5 It illustrates an example of a flowchart executed by the stressor estimation device according to the first example embodiment.

FIG. 6 It is an example of a functional block diagram of the stressor estimation device for high stress event notification process according to a second example embodiment.

FIG. 7 It is an example of a flowchart showing a procedure of a high stress event notification process according to the second example embodiment.

FIG. 8 It illustrates a schematic configuration of a stress management system in a third example embodiment.

FIG. 9 It is a block diagram of a stressor estimation device in a fourth example embodiment.

FIG. 10 It is an example of a flowchart executed by a stressor estimation device in the fourth example embodiment.

### EXAMPLE EMBODIMENTS

Hereinafter, an example embodiment of a stressor estimation device, a stressor estimation method, and a storage medium will be described with reference to the drawings.

### First Example Embodiment

### (1) System Configuration

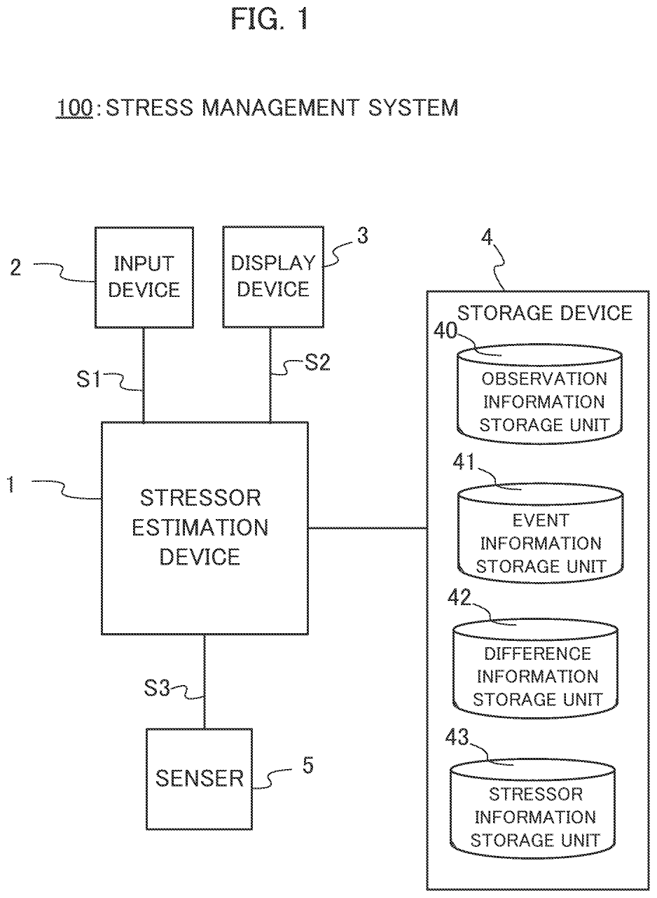

FIG. 1 shows a schematic configuration of a stress management system 100 according to the first example embodiment. The stress management system 100 manages information regarding stresses of target persons. Here, the “target person” may be a sports player or employee whose stress state is managed by an organization, or may be an individual user.

The stress management system 100 mainly includes a stressor estimation device 1, an input device 2, an output device 3, a storage device 4, and a sensor 5.

The stressor estimation device 1 estimates a stressor of a target person, and presents the estimation result. The stressor estimation device 1 performs data communication with the input device 2, the output device 3, and the sensor 5 through a communication network or through wireless or wired direct communication. For example, the stressor estimation device 1 receives an input signal “S1” supplied from the input device 2, a sensor signal “S3” supplied from the sensor 5, and various information stored in the storage device 4.

In the present example embodiment, the stressor estimation device 1 estimates the stress state (specifically, the stress value representing the degree of stress) of the target person using information (also referred to as “observation information”) based on the sensor signal S3 that is information obtained by observing (measuring) the target person. Then, the stressor estimation device 1 estimates the stressor based on the estimated stress value and event information relating to one or more events that occurred in a target period of the stress estimation. The stressor estimation device 1 generates an output control signal “S2” based on an estimation result of the stressor of the target person and supplies the generated output control signal S2 to the output device 3. In the present example embodiment, the stressor estimation device 1 calculates a short term stress value representing the degree of the short term stress that is stress in a relatively short period (e.g., a few minutes to several days), and, a chronic stress value representing the degree of the chronic stress that is stress in a longer term (e.g., a few days to a week or month) than (more chronic than) the short term stress. The term “stress reflection period” hereinafter indicates a period (i.e., period in units of several days, a week, or a month) in which the condition of the target person is reflected in the estimation of the chronic stress value.

The input device 2 is one or more interfaces configured to receive an external input (manual input) of information regarding each target person. The user who performs input of information using the input device 2 may be the target person itself, or may be a person who manages or supervises the activity of the target person. Examples of the input device 2 include a touch panel, a button, a keyboard, a mouse, a voice input device, and any other interfaces for external input. The input device 2 supplies the input S1 generated based on the input from the user to the stressor estimation device 1. The output device 3 display or output by audio information, based on the output control signal S2 supplied from the stressor estimation device 1. Examples of the output device 3 include a display, a virtual (augmented) real terminal, a projector, an audio output device such as a speaker, and any other output device.

The sensor 5 measures a biological signal or the like of the target person and supplies the measured biological signal or the like to the stressor estimation device 1 as a sensor signal S3. In this instance, the sensor signal S3 may be any biological signal (including vital information) such as heartbeat, EEG, amount of perspiration, amount of hormonal secretion, cerebral blood flow, blood pressure, body temperature, electromyogram, electrocardiogram, respiration rate, pulse wave, acceleration. The sensor 5 may be a device that analyzes blood of the target person and outputs the analysis result as a sensor signal S3. The sensor 5 may be a wearable terminal woRN by the target person, or may be a camera for photographing the target person or a microphone for generating a voice signal of the utterance of the target person. The sensor 5 may be a terminal such as a personal computer and a smartphone operated by the target person. For example, the above-described wearable terminal includes a GNSS (global navigation satellite system) receiver, an accelerometer, or any other sensor that detects a biological signal, and outputs the output signals from the respective sensors as the sensor signal S3. The sensor 5 may supply information corresponding to the operation quantity of the personal computer or the smart phone to the stressor estimation device 1 as the sensor signal S3. The sensor 5 may also output the sensor signal S3 representing biomedical data (including sleeping time information) measured from the target person during the sleep of the target person.

The storage device 4 is one or more memories which store various information necessary for estimating the stress state. The storage device 4 may be an external storage device, such as a hard disk, connected or embedded in the stressor estimation device 1, or may be a storage medium, such as a flash memory. The storage device 4 may be a server device that performs data communication with the stressor estimation device 1. Further, the storage device 4 may be configured by a plurality of devices.

The storage device 4 includes an observation information storage unit 40, an event information storage unit 41, a difference information storage unit 42, and a stressor information storage unit 43.

The observation information storage unit 40 stores observation information which is objective information regarding the target person based on the sensor signal S3. Here, the sensor signal S3 itself may be used as observation information, the features (including an index value representing the facial expression, emotion, and the like analyzed from an image or audio data) calculated based on the sensor signal S3 may be used as observation information. Further, the observation information may include questionnaire answer information based on the input signal S1 or a diagnosis result regarding a personality based on the questionnaire answer information. For examples, the observation information is stored in the observation information storage unit 40 in association with the identification information (target person ID) regarding the target person to be observed and the observation date and time information.

The event information storage unit 41 stores event information relating to the target person. For example, the event information at least includes information regarding the details (content) of the event and the date and time (time period) when the event is performed. It is noted that the event information storage unit 41 may be a system which manages the schedule of the target person or may be a device which stores, as event information, the schedule information regarding the target person acquired from the system.

The difference information storage unit 42 stores the difference information calculated by the stressor estimation device 1. The difference information includes information regarding the difference value between the chronic stress values obtained in different stress reflection periods and information regarding the difference between events which occurred in the different stress reflection periods, and it is suitably used for estimation of the stressor. The details of the difference information will be described later. The difference information is stored in the difference information storage unit 42 in association with the identification information regarding the target person, for example.

The stressor information storage unit 43 stores stressor information representing the stressor (more specifically, the cause of the chronic stress) of the target person estimated by the stressor estimation device 1. The stressor is identified, for example, by the type of the event, the time period of the event, the person who participated in the event, the place of the event, or a combination thereof. For example, the stressor information is stored in the stressor information storage unit 43 in association with the identification information regarding the target person.

The configuration of the stress management system 100 shown in FIG. 1 is an example, and various changes may be made to the configuration. For example, the input device 2 and the output device 3 may be configured integrally. In this case, the input device 2 and the output device 3 may be configured as a tablet-type terminal that is incorporated into or separate from the stressor estimation device 1. Further, the input device 2 and the sensor 5 may be configured integrally. Further, the stressor estimation device 1 may be configured by a plurality of devices. In this case, the plurality of devices constituting the stressor estimation device 1 transmits and receives information necessary for executing the preassigned process among the plurality of devices. In this case, the stressor estimation device 1 functions as an information processing system.

### (2) Hardware Configuration of Stressor Estimation Device

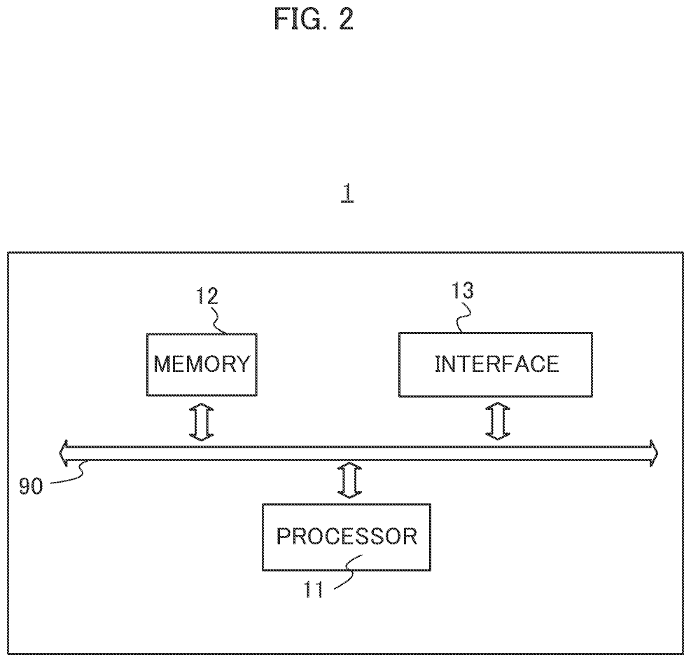

FIG. 2 shows a hardware configuration of the stressor estimation device 1. The stressor estimation device 1 includes a processor 11, a memory 12, and an interface 13 as hardware. The processor 11, memory 12 and interface 13 are connected to one another via a data bus 90.

The processor 11 functions as a controller (calculator) configured to perform overall control of the stressor estimation device 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile and non-volatile memories, such as a RAM (Random Access Memory), a ROM (Read Only Memory), and a flash memory. Further, a program for executing a process executed by the stressor estimation device 1 is stored in the memory 12. A part of the information stored in the memory 12 may be stored in one or more external storage devices capable of communicating with the stressor estimation device 1 or may be stored in a removable storage medium detachable from the stressor estimation device 1.

The interface 13 is one or more interfaces for electrically connecting the stressor estimation device 1 to other devices. Examples of the interfaces include a wireless interface, such as a network adapter, for transmitting and receiving data to and from other devices wirelessly, and a hardware interface, such as a cable, for connecting to other devices.

The hardware configuration of the stressor estimation device 1 is not limited to the configuration shown in FIG. 2. For example, the stressor estimation device 1 may include at least one of the input device 2 and/or the output device 3. The stressor estimation device 1 may be connected to or incorporate an audio output device such as a speaker.

### (3) Stressor Estimation Process

Next, a stressor estimation process that is executed by the stressor estimation device 1 will be described. In summary, the stressor estimation device 1 generates difference information representing both of the difference between the chronic stress values observed in different stress reflection periods and difference between the events which occurred in the different stress reflection periods, and estimates the stressor based on the difference information. Thus, the stressor estimation device 1 estimates the stressor of the target person with high accuracy and presents the estimation result.

### (3-1) Functional Blocks

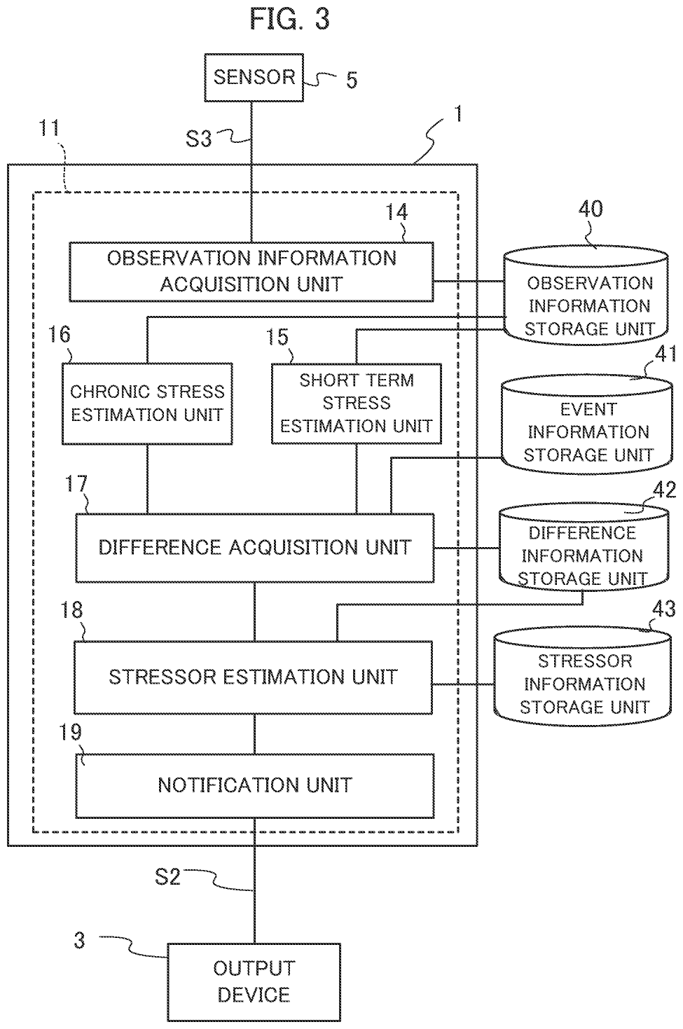

FIG. 3 is an example of a functional block diagram of the stressor estimation device 1. The processor 11 of the stressor estimation device 1 functionally includes an observation information acquisition unit 14, a short term stress estimation unit 15, a chronic stress estimation unit 16, a difference acquisition unit 17, a stressor estimation unit 18, and a notification unit 19. In FIG. 3, any blocks to exchange data with each other are connected by a solid line, but the combination of blocks to exchange data with each other is not limited to FIG. 3. The same applies to the drawings of other functional blocks described below.

Based on the sensor signal S3, the observation information acquisition unit 14 acquires the observation information regarding the target person and stores the acquired observation information in the observation information storage unit 40. In this instance, as described above, the observation information acquisition unit 14 may acquire the sensor signal S3 as the observation information, or may acquire the features (including an index value representing the facial expression, emotion, and the like analyzed from an image or audio data) calculated based on the sensor signal S3 as the observation information. The observation information acquisition unit 14 may acquire the questionnaire answer information based on the input signal S1 or the diagnosis result regarding the personality based on the questionnaire answer information as the observation information. Similarly, based on the input signal S1, or based on the sensor signal S3, the observation information acquisition unit 14 may acquire attribute(s) (e.g., age, gender, race, and occupation) of the target person as the observation information. In this case, the target person's attributes or the like are used as input information to be inputted to the short term stress estimation unit 15 or the chronic stress estimation unit 16.

The short term stress estimation unit 15 estimates the short term stress value of the target person. In this case, the short term stress estimation unit 15 extracts the observation information having a correlation with the short term stress value from the observation information storage unit 40, and estimates the short term stress value from the extracted observation information. In this case, for example, the short term stress estimation unit 15 estimates the short term stress value from acceleration, biological data, other observation information (including facial expression information) and the like. In this case, the short term stress estimation unit 15 may estimate the short term stress value using the estimation model configured to estimate a short term stress value. Here, the above-described estimation model is, for example, a learning model which is trained in advance to output a short term stress value of a target person when observation information regarding the target person is inputted thereto, and parameters for configuring the estimation model are previously stored in the storage device 4 or the like. The short term stress estimation unit 15 supplies the estimation result to the difference acquisition unit 17.

The chronic stress estimation unit 16 estimates the chronic stress value of the target person. In this case, the chronic stress estimation unit 16 extracts, from the observation information storage unit 40, the observation information that is the observation information observed in the stress reflection period and has correlation with the chronic stress value, and then estimates the chronic stress value from the extracted observation information. In this case, the chronic stress estimation unit 16 may estimate the chronic stress value from the observation information based on any method. In addition, the chronic stress estimation unit 16 may estimate the chronic stress value from the observation information (that is, subjectively measured information) based on the answer result of a questionnaire for estimating the chronic stress. Examples of the questionnaire for estimating chronic stress values include the PSS (Perceived Stress Scale) questionnaire. In addition, the chronic stress estimation unit 16 may estimate the chronic stress value using an estimation model configured to estimate a chronic stress value. The chronic stress estimation unit 16 supplies the estimation result to the stressor estimation unit 18.

The short term stress estimation unit 15 and the chronic stress estimation unit 16 may calculate the stress values at predetermined time intervals, or may collectively calculate the stress values necessary for estimating the stressor when it is determined to be an estimation timing of the stressor. The short term stress estimation unit 15 and the chronic stress estimation unit 16 may store the stress estimation results in the storage device 4 or the like instead of supplying the stress estimation results to the difference acquisition unit 17. In this case, the difference acquisition unit 17 acquires the stress values necessary for calculating the difference information from the storage device 4.

The difference acquisition unit 17 generates difference information representing the difference between two chronic stress values obtained in different stress reflection periods and the difference between the events which occurred in the different stress reflection periods, based on the stress estimation results by the short term stress estimation unit 15 and the chronic stress estimation unit 16 and the event information regarding the target person extracted from the event information storage unit 41. In this case, for example, based on the stress estimation result by the short term stress estimation unit 15, the difference acquisition unit 17 generates the difference information obtained by using selected events in which the short term stress value is equal to or larger than a threshold value. The difference acquisition unit 17 generates plural pieces of difference information by sliding (shifting) the stress reflection period. Details on how to generate differential information will be described later. The difference acquisition unit 17 stores the generated difference information in the difference information storage unit 42. The difference acquisition unit 17 may generate a necessary amount (e.g., a predetermined number of pieces) of difference information when receiving a notification from the stressor estimation unit 18 to estimate the stressor, or may generate the difference information at predetermined time intervals (e.g., at each timing when the chronic stress estimation unit 16 calculates the chronic stress value).

The stressor estimation unit 18 estimates the stressor relating to the chronic stress of the target person based on the plural pieces of difference information calculated by the difference acquisition unit 17. In this case, for example, the stressor estimation unit 18 estimates the stressor (e.g., the type, place, participant, and/or time period of the event) using any machine learning method or statistical method such as a correlation analysis, a partial correlation analysis, and a principal component analysis. Then, the stressor estimation unit 18 supplies the stressor information representing the estimated stressor to the notification unit 19, and stores the stressor information in the stressor information storage unit 43 in association with the identification information regarding the target person.

The notification unit 19 performs a control of the output device 3 to output information regarding the stressor estimated by the stressor estimation unit 18. In this instance, the notification unit 19 generates an output control signal S2 for displaying or outputting, by audio, information on the stressor, and supplies the output control signal S2 to the output device 3 via the interface 13. In this case, for example, the notification unit 19 outputs information to notify the user of the type, place, participant or/and time period of the event(s) that falls under the stressor to the output device 3.

In general, if a part of the short term stress leading to an increase in the chronic stress is accumulated without relieving it, the chronic stress increases. Taking the above into consideration, the stressor estimation device 1 estimates the stressor that is liable to become chronic stress, and notifies the user of the estimation result. This enables users to recognize tendencies such as types, time periods, and places of events in which stress is especially apt to become chronic, and encourage users to take appropriate measures to deal with them.

Each component of the observation information acquisition unit 14, the short term stress estimation unit 15, the chronic stress estimation unit 16, the difference acquisition unit 17, the stressor estimation unit 18, and the notification unit 19 described in FIG. 3 can be realized by the processor 11 executing a program, for example. Additionally, the necessary programs may be recorded on any non-volatile storage medium and installed as necessary to realize each component. It should be noted that at least a portion of each of these components may be implemented by any combination of hardware, firmware, and software, without being limited to being implemented by software based on a program. At least some of these components may also be implemented using user programmable integrated circuit such as, for example, a FPGA (Field-Programmable Gate Array) and a microcontroller. In this case, the integrated circuit may be used to realize a program functioning as each of the above components. Further, at least a part of the components may be configured by ASSP (Application Specific Standard Produce), ASIC (Application Specific Integrated Circuit) or a quantum processor (quantum computer control chip). Thus, each of the above-described components may be realized by various hardware. Furthermore, each of these components may be implemented by cooperation of a plurality of computers, for example, using cloud computing technology.

### (3-2) Generation of Difference Information

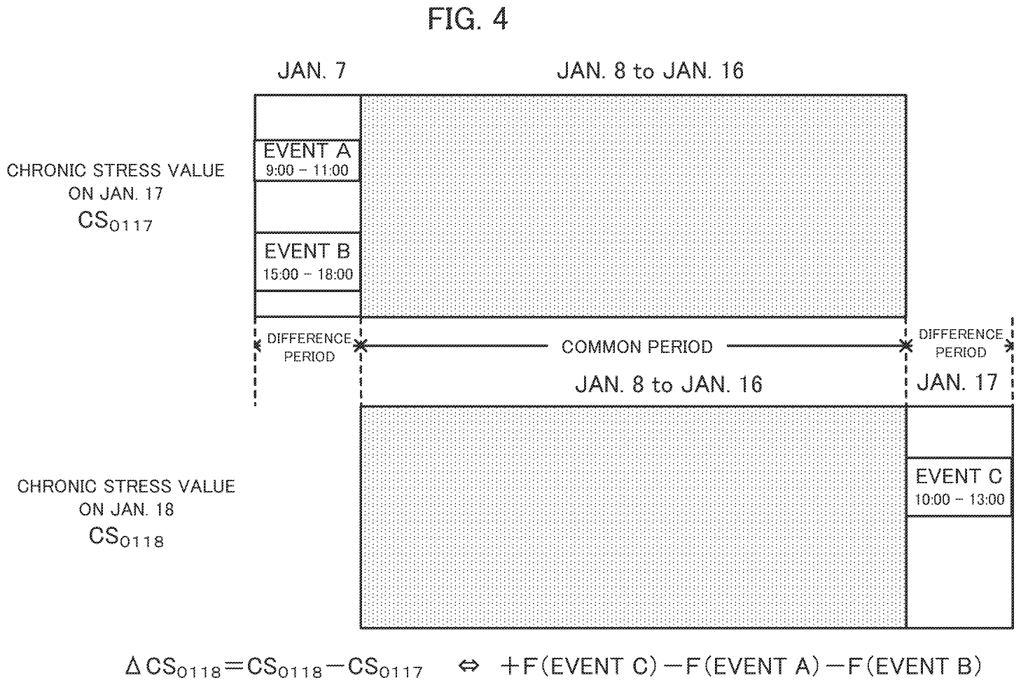

FIG. 4 is a diagram showing a specific example of the generation of the difference information by the difference acquisition unit 17. FIG. 4 illustrates the schedule of the target person and an equation equivalent to the difference information to be described later in the stress reflection periods corresponding to the chronic stress value “CS₀₁₁₇” on January 17 and the chronic stress value “CS₀₁₁₈” on January 18 on the next day. It shows a specific example of the generation of difference information based on the difference. Here, the period from January 8 to January 16 is a period (also referred to as “common period”) in which the stress reflection periods overlaps, and January 7 and January 17 are non-overlapping periods (also referred to as “difference periods”) of the stress reflection periods. It is noted that the schedule of the target person in the common period is not explicitly shown in FIG. 4 because it is not related to the generation of the difference information.

In the example shown in FIG. 4, when calculating the chronic stress value of a certain target day, the chronic stress estimation unit 16 determines the stress reflection period to be a period from 10 days before the target day to the day before the target day, and calculates the chronic stress value of the target day based on the observation information in the determined stress reflection period. Then, the chronic stress estimation unit 16 calculates the chronic stress value everyday while shifting the target day by one day. Specifically, the chronic stress estimation unit 16 calculates the chronic stress value CS₀₁₁₇ of January 17 based on the observation information observed in the stress reflection period from January 7 to January 16, and calculates the chronic stress value CS₀₁₁₈ of January 18 based on the observation information observed in the stress reflection period from January 8 to January 17.

Then, the difference acquisition unit 17 generates the difference information for each pair of the chronic stress values whose stress reflection periods are deviated by one day from each other. In the exemplary example shown in FIG. 4, the difference acquisition unit 17 generates the difference information for the chronic stress values CS₀₁₁₇ and CS₀₁₁₈ whose stress reflection periods are deviated by one day from each other.

When generating the difference information for the chronic stress values CS₀₁₁₇ and CS₀₁₁₈, the difference acquisition unit 17 first acquires the event information regarding the events in the stress reflection periods of the chronic stress values CS₀₁₁₇ and CS₀₁₁₈ from the event information storage unit 41, respectively. The difference acquisition unit 17 identifies an event (also referred to as “high stress event”) in which the short term stress value of the target person becomes equal to or larger than a threshold value among the events that occur in the difference periods of the stress reflection periods which there is no overlap therebetween. For example, the above threshold value (also referred to as “event determination threshold value”) is stored in the storage device 4 or the like in advance. In the example shown in FIG. 4, among the events which occurred in January 7 and January 17, which are non-overlapping difference periods, the difference acquisition unit 17 identifies, as high stress events, the event A, event B, and event C in which the short term stress values in the occurrence time period of the events are equal to or larger than the event determination threshold value. Examples of the events A to C include various events such as “monthly report”, “visit to client”, and “internal meeting”.

It is noted that the short term stress value determined per event to be used for determination of a high stress event may be any representative value (statistical value) such as the average, median, and maximum value of the short term stress values acquired during the execution period of a target event, or may be the short term stress value at the end of the target event.

Then, the difference acquisition unit 17 determines that the difference “ΔCS₀₁₁₈” (=CS₀₁₁₈−CS₀₁₁₇” between the chronic stress value CS₀₁₁₇ and the chronic stress value CS₀₁₁₈ occurred due to the high stress events in the difference periods. Provided that “F(x)” denote the increase in the chronic stress caused by a certain event x, the difference ΔCS₀₁₁₈ is expressed by the following equation (1).

ΔCS₀₁₁₈≈+F(Event C)−F(Event A)−F(Event B) (1)

In the equation (1), the sign of the event C, which exists only in the stress reflection period of the chronic stress value CS₀₁₁₈ subject to subtraction in the calculation of the difference ΔCS₀₁₁₈, is set to be positive (+), and the signs of the event A and the event B, which exist only in the stress reflection period of the chronic stress value CS₀₁₁₇ which is subtracted in the calculation of the difference ΔCS₀₁₁₈, are each set to be negative (−).

The equation (1) shows the relation between: the difference between the chronic stress values; and the high stress events that bring about the difference. It provides information that is suitable for estimating stressors. For example, if ΔCS₀₁₁₈ is a positive value, event C can be considered to be an event that has a higher correlation with the chronic stress value than the event A and the event B.

Then, the difference acquisition unit 17 generates information representing the relation (the equation (1) in the case shown in FIG. 4) between: the difference between the chronic stress values; and the high stress events each of which causes the difference as the difference information corresponding to the chronic stress values CS₀₁₁₇ and CS₀₁₁₈. Specifically, the difference information at least includes the difference ΔCS₀₁₁₈ between the chronic stress values corresponding to the left side of the equation (1) and the combinations of the types of the events and signs of the events which correspond to terms in the right side of the equation (1).

If there are terms of events which fall under the same type and have different signs, in the right side of the equation (1), the difference acquisition unit 17 may simplify the equation by integrating these terms. If there are terms “+F (Event A)” and “−F (Event A)”, the difference acquisition unit 17 cancels these terms. If there are terms “+2F(Event A)” and “−F (Event A)”, the difference acquisition unit 17 sets “+F (Event A)” as the integration result.

In some embodiment, the difference acquisition unit 17 may include information representing the short term stress value corresponding to each high stress event in the difference information. In this case, the type, the sign (positive/negative) and the short term stress value of each high stress event are used for estimating the stressor by the stressor estimation unit 18. The information representing the short term stress value may be information representing the short term stress value itself, or may be information representing the level of the short term stress value.

As described above, the difference acquisition unit 17 generates difference information equivalent to the difference between the chronic stress values observed in the stress reflection periods which are deviated from each other by one day and the difference between the events which occurred in the stress reflection periods. Thus, the difference acquisition unit 17 can suitably generate the difference information necessary for the estimation of the stressor. In the example shown in FIG. 4, the pair of the chronic stress values CS₀₁₁₇ and CS₀₁₁₈ is an example of a pair of “the first chronic stress value” and “the second chronic stress value”. The stress reflection period of the chronic stress value CS₀₁₁₇ (from January 7 to January 16) and the stress reflection period of the chronic stress value CS₀₁₁₈ (from January 8 to January 17) are examples of “the first period” and “the second period”, respectively.

### (3-3) Estimation of Stressors

Next, a description will be given of a method of estimating a stressor by the stressor estimation unit 18. The stressor estimation unit 18 estimates the type, place, participant, and/or time period and the like of each event that becomes a stressor of the target person based on plural pieces of the difference information regarding the target person.

For example, according to an arbitrary correlation analysis (partial correlation analysis) method, the stressor estimation unit 18 calculates the correlation (correlation coefficients or partial correlation coefficients) between each high stress event and the chronic stress value, based on the relation between: the difference between the chronic stress values indicated by plural pieces of the difference information; and the high stress event. In some embodiments, the stressor estimation unit 18 may calculate the above-described correlation using the short term stress values of the high stress events. Then, the stressor estimation unit 18 determines that the higher the calculated correlation of a high stress event is, the higher the adequacy of the high stress event as the stressor becomes, and therefore specifies high stress events whose correlations are equal to or larger than a predetermined value. Then, the stressor estimation unit 18 aggregates the type, place, participant, and/or time period of each specified high stress event and estimates the stressor based on the aggregation result. For example, the stressor estimation unit 18 identifies, as the stressors, the event type, event place, event participant, and/or event time period whose proportion is equal to or larger than a predetermined proportion among high stress events whose correlations are equal to or larger than the predetermined value. It is noted that, instead of the above-described examples, the stressor estimation unit 18 may identify an event that contributes to an increase in the chronic stress value based on an arbitrary multivariate analysis method or an arbitrary machine learning, such as a cluster analysis, a principal component analysis, a vector quantization, and a self-organizing map (SOM).

### (3-4) Processing Flow

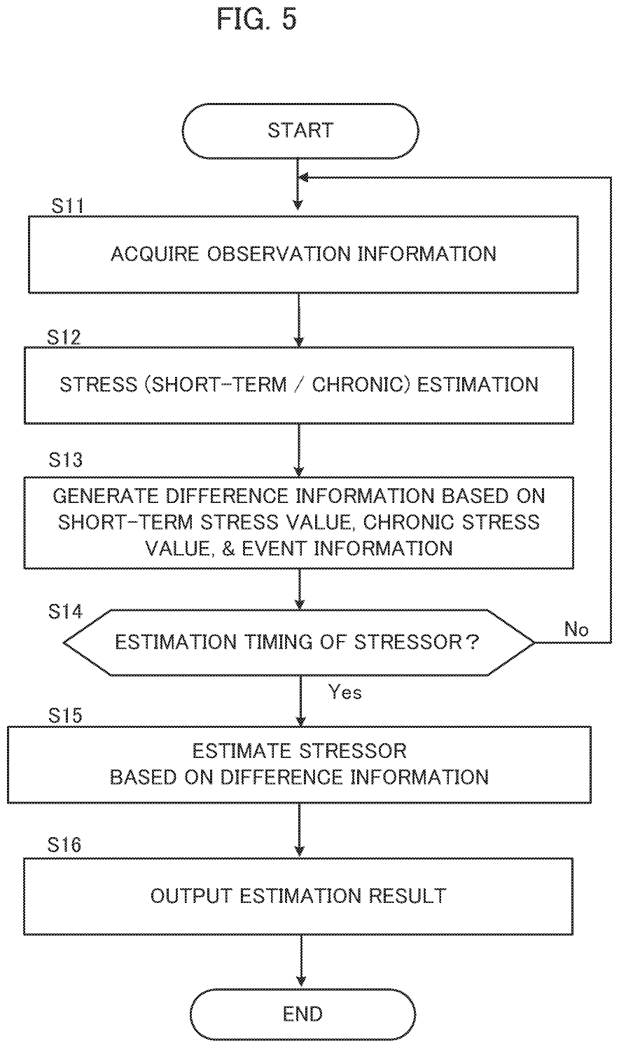

FIG. 5 is an example of a flowchart that is executed by the stressor estimation device 1 in the first example embodiment. For example, the stressor estimation device 1 repeatedly executes the processing of the flowchart shown in FIG. 5. In FIG. 5, as an example, the stressor estimation device 1 calculates the short term stress value, the chronic stress value, and the difference information at intervals of predetermined times that are set to be respective time lengths, and estimates the stressor and outputs the estimation result at the estimation timing of the stressor.

First, the stressor estimation device 1 acquires the observation data (step S11). In this instance, the stressor estimation device 1 acquires the observation information on the basis of the sensor signal S3 supplied from the sensor 5 and stores the acquired observation information in the observation information storage unit 40.

Next, the stressor estimation device 1 estimates the short term stress value and the chronic stress value based on the observation information stored in the observation information storage unit 40 (step S12). In this case, for example, the stressor estimation device 1 estimates the short term stress value at an interval of a first time (e.g., several minute intervals), and estimates the chronic stress value at an interval of a second time (e.g., one day intervals) which is longer than the interval of the first time. The stressor estimation device 1 stores these estimation results in the storage device 4 or the like.

Then, the stressor estimation device 1 generates difference information based on the short term stress value, the chronic stress value, and the event information (step S13). In this instance, for example, the stressor estimation device 1 generates the difference information at the same second time intervals as the estimation of the chronic stress value at step S12, and stores the generated difference information in the difference information storage unit 42. In this case, for example, each time the chronic stress value is estimated, the stressor estimation device 1 generates difference information corresponding to the difference between the latest estimated chronic stress value and the second latest estimated chronic stress value.

Next, the stressor estimation device 1 determines whether or not the estimation timing of the stressor has come up (step S14). For example, the stressor estimation device 1 determines the estimation timing of the stressor has come up if the stressor estimation device 1 detects instructions to execute the stressor estimation based on the input signal S1 supplied from the input device 2, or, if a predetermined time has elapsed from the previous estimation timing of the stressor.

Then, if it is determined that the estimation timing of the stressor has come up (step S14; Yes), the stressor estimation device 1 estimates the stressor relating to the chronic stress of the target person based on the difference information regarding the target person generated at step S13 (step S15). Then, the stressor estimation device 1 outputs the estimation result of the stressor generated at step S15 (step S16). In this case, for example, the stressor estimation device 1 supplies the output control signal S2 to the output device 3 to thereby cause the output device 3 to display or output, by audio, information on the type, place, participants, time period and the like of the event(s) estimated as the stressor.

The stressor estimation device 1 may perform at least one of the process at step S12 and the process at step S13 collectively after determining that the stressor estimation timing has come up at step S14.

### (4) Modifications

Next, a description will be given of modifications of the example embodiment described above. The following modifications may be applied to the example embodiments described above in any combination.

### (First Modification)

Instead of generating the difference information based on a pair of the chronic stress values whose stress reflection periods are deviated from each other by one day, the difference acquisition unit 17 may generate the difference information based on a pair of the chronic stress values whose stress reflection periods are deviated from each other by more than one day.

For example, in a case where the difference acquisition unit 17 generates the difference information on the basis of a pair of chronic stress values whose stress reflection periods are deviated from each other by X (X is an integer of 2 or more) days, the difference acquisition unit 17 acquires event information corresponding to the difference periods included in the stress reflection periods, and identifies high stress events in the difference periods on the basis of the short term stress values. Then, the difference acquisition unit 17 generates difference information regarding the difference between the chronic stress values and the difference between the high stress events.

As described above, the difference acquisition unit 17 can suitably generate the difference information for the pair of the chronic stress values which is the stress reflecting period deviated by two days or more. Then, the stressor estimation unit 18 estimates the stressor based on the difference information generated in this way, and thereby can estimate a combination of a plurality of causes of stress deterioration.

In this modification, the above-described X day may be longer than the stress reflection period of the chronic stress value. In this case, the stress reflection periods corresponding to a pair of chronic stress values are periods without any overlapping with each other.

### (Second Modification)

In the determination of the high stress event, the difference acquisition unit 17 may calculate the increase in the short term stress value from the start time to the end time of an event of interest, and determine that the event in which the calculated increase is equal to or more than a predetermined threshold value is a high stress event. According to this mode, the difference acquisition unit 17 can accurately identify the event in which the short term stress value of the target person has increased during the event as a high stress event.

### (Third Modification)

The difference acquisition unit 17 may identify an event in which the amount of decrease in the short term stress value from the start time of the event to the end time of the event is equal to or more than a predetermined value, as an event (also referred to as “stress relieving event”) that is effective in relieving the stress.

In this case, for example, the difference acquisition unit 17 extracts, among the events belonging to the difference periods, one or more events in which the amount of decrease in the short term stress value is equal to or more than a predetermined threshold value as stress relieving event. Then, the difference acquisition unit 17 generates difference information indicating the type and the sign of the stress relieving event.

Then, the stressor estimation unit 18 estimates, as a stressor, the cause of relieving the stress. In this case, the stressor estimation unit 18 calculates the correlation between the chronic stress value and each stress relieving event, and aggregates types, places, participants, and/or time periods of stress relieving events in which the correlation is negative and the absolute value of the correlation is equal to or more than a predetermined value. Then, the stressor estimation unit 18 estimates the event type, event place, event participants or/and event time period that are causes of relieving the stress, based on the aggregation result. Then, the notification unit 19 controls the output device 3 to output the information regarding the causes of relieving the stress estimated by the stressor estimation unit 18.

In this way, according to this modification, the stressor estimation device 1 can suitably estimate the causes of relieving the stress and notify the user of the estimation result. The stressor estimation device 1 may estimate, in addition to the process according to this modification, the causes of boosting the stress based on the example embodiment described above, and control the output device 3 to output both of the causes of relieving the stress and the causes of boosting the stress.

### Second Example Embodiment

In addition to the stressor estimation process described in the first example embodiment, the stress management system 100 according to the second example embodiment detects the occurrence of a high stress event based on the estimated stressor and performs a high stress event notification process for giving the user a notification to encourage the user to relieve the stress based on the detection result. Such a high stress event notification process suitably suppresses the chronic stress generation by encouraging the user to relieve the stress when an event which tends to bring about the chronic stress occurs. The same components in the second example embodiment as those in the first example embodiment are appropriately denoted by the same reference numerals, and a description thereof will be omitted.

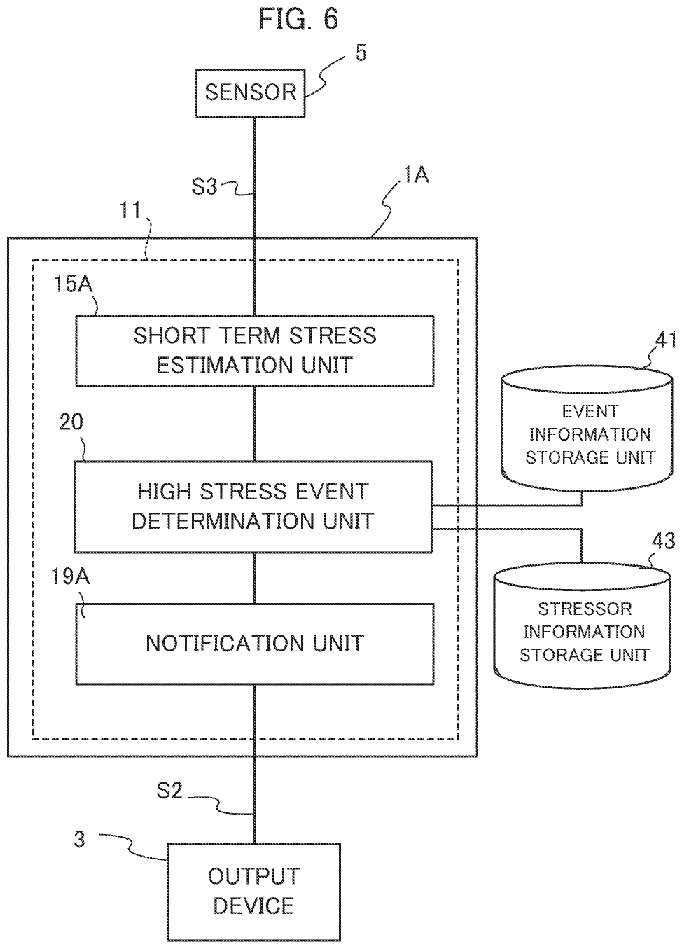

FIG. 6 is a functional block diagram illustrating a high stress event notification process executed by the stressor estimation device TA according to the second example embodiment. The stressor estimation device TA according to the second example embodiment is equipped with the hardware configuration shown in FIG. 2, and the processor 11 of the stressor estimation device TA has the function block relating to the high stress event notification process shown in FIG. 6 in addition to the function block relating to the stressor estimation process shown in FIG. 3. As shown in FIG. 6, the processor 11 includes a short term stress estimation unit 15A, a high stress event determination unit 20, and a notification unit 19A in relation to the high stress event notification process. It is herein assumed that the stressor information regarding the target person is already stored in the stressor information storage unit 43 through the stressor estimation process described in the first example embodiment.

The short term stress estimation unit 15A estimates the short term stress value of the target person observed by the sensor 5 based on the sensor signal S3 supplied from the sensor 5. In this instance, the short term stress estimation unit 15A estimates the short term stress value of the target person at the present time from the sensor signal S3 through processes executed by the observation information acquisition unit 14 and the short term stress estimation unit 15 according to the first example embodiment.

The high stress event determination unit 20 determines the high stress event based on the event information regarding the target person stored in the event information storage unit 41 and the stressor information regarding the target person stored in the stressor information storage unit 43. In this case, for example, the high stress event determination unit 20 determines that a high stress event has occurred, if an ongoing event identified based on the above-described event information and the present date and time is an event falling under the stressor indicated by the stressor information regarding the target person, and, the corresponding short term stress value is equal to or larger than the event determination threshold value. In this case, the above-mentioned event determination threshold value is identical to the event determination threshold value used in determining the high stress event from the short term stress value in the first example embodiment, and is previously stored in the storage device 4 or the like. In addition, “the event falling under the stressor” refers to an event which matches the event type, event place, event participants, and/or event time indicated by the stressor information. The high stress event determination unit 20 supplies the determination result of the high stress event to the notification unit 19A.

The notification unit 19A controls the output device 3 to output information regarding the determination result of the high stress event generated by the high stress event determination unit 20. In this case, for example, when the high stress event determination unit 20 determines that a high stress event has occurred, the notification unit 19A causes the output device 3 through the output control signal S2 to output the information indicating that the high stress event has occurred and information prompting the user to relieve the stress.

The stressor estimation device 1A can accurately detect the occurrence of a high stress event that could bring about the chronic stress through such a high stress event notification process, and suitably prompt the user to eliminate the stress so that the chronic stress level does not increase due to the occurrence of the high stress event.

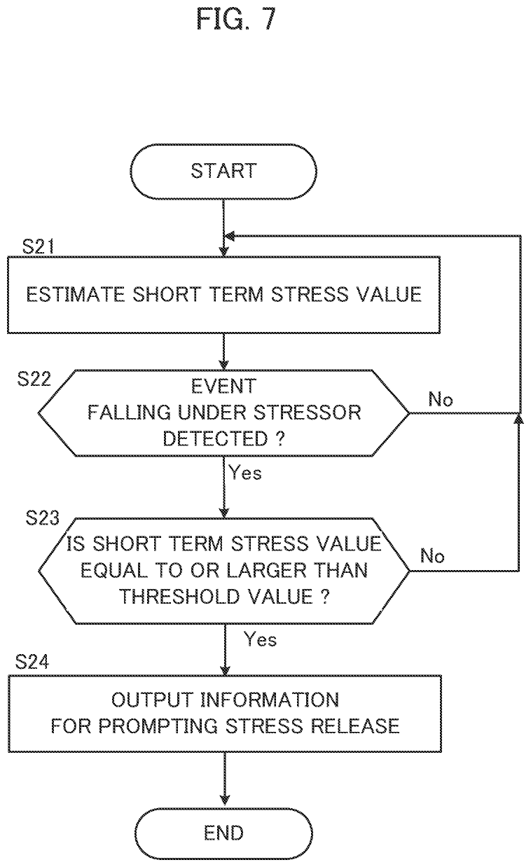

FIG. 7 is an example of a flowchart illustrating a procedure of the high stress event notification process in the second example embodiment. The stressor estimation device 1A repeatedly executes the process of the flowchart shown in FIG. 7.

First, the stressor estimation unit 1A estimates the short term stress of the target person based on the sensor signal S3 supplied from the sensor 5 (step S21).

Next, the stressor estimation device 1A determines whether or not an event falling under the stressor according to the stressor information regarding the target person stored in the stressor information storage unit 43 is detected (step S22). Then, if the stressor estimation device 1A detects an event falling under the above-described stressor (step S22; Yes), it determines whether or not the short term stress value calculated at step S21 is equal to or larger than the stress determination threshold value (step S23). In this case, the stressor estimation device 1A may adopt any short term stress value estimated during the event falling under the stressor as the short term stress value to be compared to the stress determination threshold value, or may adopt the average value or any other representative value (statistical value) of a plurality of short term stress values estimated during the event.

if an event falling under the stressor is detected (step S22; Yes) and the short term stress value is equal to or larger than the stress determination threshold value (step S23; Yes), the stressor estimation device 1A causes the output device 3 to output the information prompting the stress release (step S24). Thus, the stressor estimation device 1A can favorably suppress the chronic stress by prompting the target person to relieve the stress when detecting an event that tends to cause chronic stress.

On the other hand, if any event falling under the stressor is not detected (step S22; No), or if the short term stress value is less than the stress determination threshold value (step S22; No), the stressor estimation device 1 gets back to the process at step S21.

### Third Example Embodiment

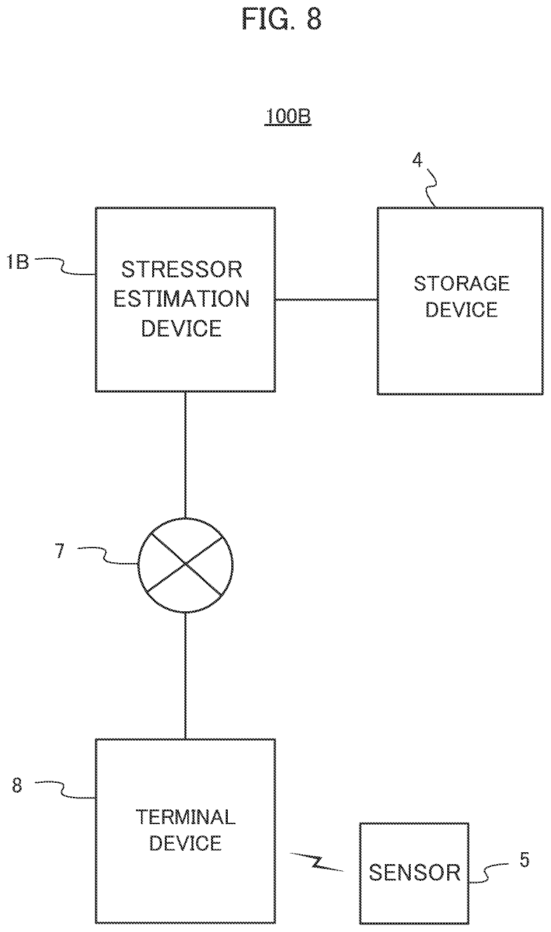

FIG. 8 shows a schematic configuration of a stress management system 100B according to the third example embodiment. The stress management system 100B according to the third example embodiment is a server client model system, and a stressor estimation device 1B functioning as a server device performs the process executed by the stressor estimation device 1A in the first example embodiment or the stressor estimation device 1 in the second example embodiment. Hereinafter, the same components as in the first example embodiment or the second example embodiment are appropriately denoted by the same reference numerals, and a description thereof will be omitted.

As shown in FIG. 8, the stress management system 100B mainly includes a stressor estimation device 1B that functions as a server, a storage device 4, and a terminal device 8 that functions as a client. The stressor estimation device 1B and the terminal device 8 perform data communication with each other via the network 7.

The terminal device 8 is a terminal used by a user to be a target person, and is equipped with an input function, a display function, and a communication function, and therefore functions as the input device 2 and the output device 3 shown in FIG. 1. Examples of the terminal device 8 may include a personal computer, a tablet-type terminal such as a smartphone, and a PDA (Personal Digital Assistant). The terminal device 8 is electrically connected to the sensor 5 such as the wearable sensor worn by the user, and transmits a biological signal or the like (i.e., information corresponding to the sensor signal S3 in FIG. 1) of the target person outputted by the sensor 5 to the stressor estimation device 1B. Further, the terminal device 8 transmits the information (information corresponding to the input signal S1 in FIG. 1) generated by an external input to the stressor estimation device 1B.

The stressor estimation device 1B is equipped with the same hardware configuration as the hardware configuration of the stressor estimation device 1 shown in FIG. 2, and the processor 11 of the stressor estimation device 1B is equipped with the functional blocks shown in FIG. 3. Then, the stressor estimation device 1B receives the information corresponding to the input signal S1 and the sensor signal S3 in FIG. 1 from the terminal device 8 via the network 7, and executes the stressor estimation process (and high stress event notification process). The stressor estimation device 1B transmits an output signal for outputting the stress estimation result to the terminal device 8 through the network 7 in response to the display request from the terminal device 8.

According to the third example embodiment, it is possible to estimate the stressor of the target person based on the biological signal or the like of the target person received from the terminal device 8 used by the target person, and suitably notify the target person of the estimation result and the occurrence of and the high stress event by the terminal device 8.

### Fourth Example Embodiment

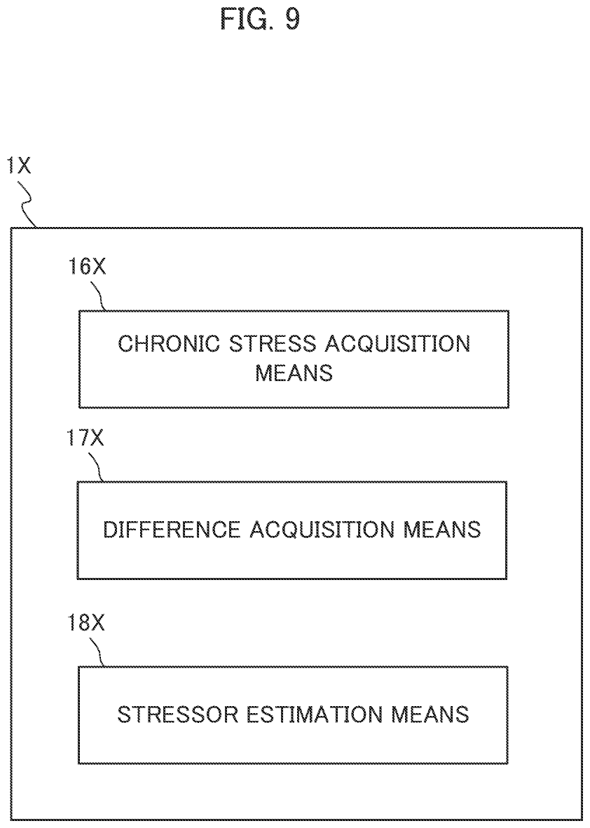

FIG. 9 is a block diagram of a stressor estimation device 1X according to the fourth example embodiment. The stressor estimation device 1X mainly includes a chronic stress acquisition means 16X, a difference acquisition means 17X, and a stressor estimation means 18X. The stressor estimation device 1X may be configured by a plurality of devices.

The chronic stress acquisition means 16X is configured to acquire a first chronic stress value of a target person in a first period and a second chronic stress value of the target person in a second period. In this instance, the chronic stress acquisition means 16X may be configured to acquire the first chronic stress value and the second chronic stress value by calculating the first chronic stress value and the second chronic stress value from a biomedical signal of the target person or the like, or may be configured to acquire the first chronic stress value and the second chronic stress value stored in the storage device or the like or calculated by another device. Examples of the chronic stress acquisition means 16X include the chronic stress estimation unit 16 according to any of the first example embodiment (the same applies hereinafter) to the third example embodiment.

The difference acquisition means 17X is configured to acquire difference information representing a difference between the first chronic stress value and the second chronic stress value and a difference between an event that occurred in the first period and an event that occurred in the second period. Examples of the difference acquisition means 17X include the difference acquisition unit 17 according to any of the first example embodiment to the third example embodiment.

The stressor estimation means 18X is configured to estimate a stressor relating to a chronic stress of the target person, based on the difference information. Examples of the stressor estimation means 18X include the stressor estimation unit 18 according to any of the first example embodiment to the third example embodiment.

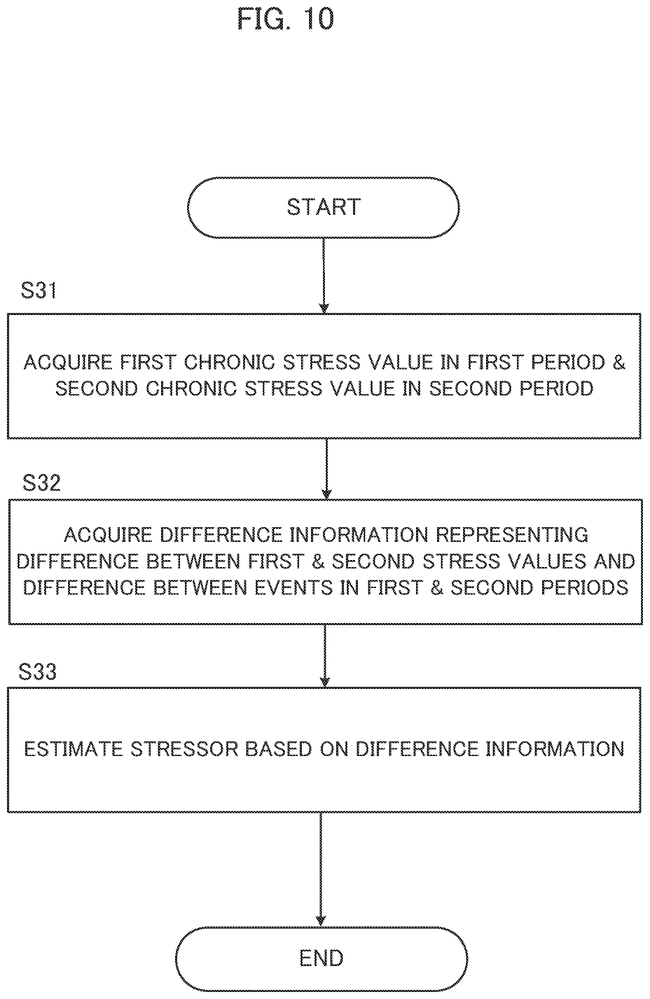

FIG. 10 is an exemplary flowchart that is executed by the stressor estimation device 1X in the fourth example embodiment. First, the chronic stress acquisition means 16X acquires a first chronic stress value of a target person in a first period and a second chronic stress value of the target person in a second period (step S31). Next, the difference acquisition means 17X acquires difference information representing a difference between the first chronic stress value and the second chronic stress value and a difference between an event that occurred in the first period and an event that occurred in the second period (step S32). Then, the stressor estimation means 18X estimates a stressor relating to a chronic stress of the target person, based on the difference information (step S33).

According to the fourth example embodiment, the stressor estimation device 1X can suitably estimate the stressor of the target person.

In the example embodiments described above, the program is stored by any type of anon-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### [Supplementary Note 1]

A stressor estimation device comprising:
a chronic stress acquisition means configured to acquire a first chronic stress value of a target person in a first period and a second chronic stress value of the target person in a second period;
a difference acquisition means configured to acquire difference information representing a difference between the first chronic stress value and the second chronic stress value and a difference between an event that occurred in the first period and an event that occurred in the second period; and
a stressor estimation means configured to estimate a stressor relating to a chronic stress of the target person, based on the difference information.

### [Supplementary Note 2]

The stressor estimation device according to Supplementary Note 1, further comprising
a short term stress acquisition means configured to acquire a short term stress value of the target person,
wherein the difference acquisition means is configured to generate the difference information representing, as the difference, an event
   which occurred in non-overlapping periods of the first period and the second period and
   in which the short term stress value or increase in the short term stress value is equal to or larger than a threshold value.

### [Supplementary Note 3]

The stressor estimation device according to Supplementary Note 1 or 2,
wherein the stressor estimation means is configured to at least estimate, as the stressor, a cause of boosting the chronic stress value of the target person.

### [Supplementary Note 4]

The stressor estimation device according to any one of Supplementary Notes 1 to 3,
wherein the stressor estimation means is configured to at least estimate, as the stressor, a cause of relieving the chronic stress value of the target person.

### [Supplementary Note 5]

The stressor estimation device according to Supplementary Note 4,
wherein the difference acquisition means is configured to generate the difference information representing, as the difference, an event
   which occurred in non-overlapping periods of the first period and the second period and
   in which a decrease in the short term stress value is equal to or larger than a threshold value.

### [Supplementary Note 6]

The stressor estimation device according to any one of Supplementary Notes 1 to 5,
wherein the stressor estimation means is configured to estimate, as the stressor, at least one of a type of the event, a place of the event, a person who participated in the event, and/or a time period of the event.

### [Supplementary Note 7]

The stressor estimation device according to any one of Supplementary Notes 1 to 6, further comprising
a notification means configured to control an output device to output information regarding the stressor estimated by the stressor estimation means.

### [Supplementary Note 8]

The stressor estimation device according to any one of Supplementary Notes 1 to 7, further comprising:
an event determination means configured to determine whether or not an event falling under the stressor estimated by the stressor estimating means has occurred; and
a notification means configured to control an output device to output information based on a determination result generated by the event determination means.

### [Supplementary Note 9]

The stressor estimation device according to Supplementary Note 8,
wherein the notification means is configured to cause the output device to output information for prompting stress release, if an event falling under the stressor has occurred and the short term stress value of the target person or an increase in the short term stress value is equal to or larger than a threshold value during the event.

### [Supplementary Note 10]

A stressor estimation method executed by a computer, the control method comprising:
acquiring a first chronic stress value of a target person in a first period and a second chronic stress value of the target person in a second period;
acquiring difference information representing a difference between the first chronic stress value and the second chronic stress value and a difference between an event that occurred in the first period and an event that occurred in the second period; and
estimating a stressor relating to a chronic stress of the target person, based on the difference information.

### [Supplementary Note 11]

A storage medium storing a program executed by a computer, the program causing the computer to:
acquire a first chronic stress value of a target person in a first period and a second chronic stress value of the target person in a second period;
acquire difference information representing a difference between the first chronic stress value and the second chronic stress value and a difference between an event that occurred in the first period and an event that occurred in the second period; and
estimate a stressor relating to a chronic stress of the target person, based on the difference information.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1. 1A, 1B, 1X Stressor estimation device
2 Input device
3 Output device
4 Storage device
5 Sensor
8 Terminal device
100, 100B Stress management system

What is claimed is:

1. A stressor estimation device comprising:
at least one memory configured to store instructions; and
at least one processor configured to execute the instructions to:
acquire observation information regardinq a target person, the observation information includinq a biological signal measured by a sensor worn by the target person;
estimate a first chronic stress value of the target person in a first period by usinq an estimation model based on the observation information observed in the first period;
estimate a second chronic stress value of the target person in a second period by usinq an estimation model based on the observation information observed in the second period;
acquire difference information representing a difference between the first chronic stress value and the second chronic stress value and a difference between an event that occurred in the first period and an event that occurred in the second period; and
estimate a stressor relating to a chronic stress of the target person, based on the difference information.

2. The stressor estimation device according to claim 1,
wherein the at least one processor is configured to further execute the instructions to acquire a short term stress value of the target person,
wherein the at least one processor is configured to execute the instructions to generate the difference information representing, as the difference, an event
which occurred in non-overlapping periods of the first period and the second period and
in which the short term stress value or increase in the short term stress value is equal to or larger than a threshold value.

3. The stressor estimation device according to claim 1,
wherein the at least one processor is configured to execute the instructions to at least estimate, as the stressor, a cause of boosting the chronic stress value of the target person.

4. The stressor estimation device according to claim 1,
wherein the at least one processor is configured to execute the instructions to at least estimate, as the stressor, a cause of relieving the chronic stress value of the target person.

5. The stressor estimation device according to claim 4,
wherein the at least one processor is configured to execute the instructions to generate the difference information representing, as the difference, an event
which occurred in non-overlapping periods of the first period and the second period and
in which a decrease in the short term stress value is equal to or larger than a threshold value.

6. The stressor estimation device according to claim 1,
wherein the at least one processor is configured to execute the instructions to estimate, as the stressor, at least one of a type of the event, a place of the event, a person who participated in the event, and/or a time period of the event.

7. The stressor estimation device according to claim 1,
wherein the at least one processor is configured to further execute the instructions to control an output device to output information regarding the stressor.

8. The stressor estimation device according to claim 1,
wherein the at least one processor is configured to further execute the instructions to make a determination of whether or not an event falling under the stressor has occurred; and
the at least one processor is configured to execute the instructions to control an output device to output information based on result of the determination.

9. The stressor estimation device according to claim 8,
wherein the at least one processor is configured to execute the instructions to cause the output device to output information for prompting stress release, if an event falling under the stressor has occurred and the short term stress value of the target person or an increase in the short term stress value is equal to or larger than a threshold value during the event.

10. A stressor estimation method executed by a computer, the control method comprising:
acquirinq observation information regardinq a target person, the observation information includinq a biological signal measured by a sensor worn by the target person;
estimatinq a first chronic stress value of the target person in a first period by usinq an estimation model based on the observation information observed in the first period;
estimatinq a second chronic stress value of the target person in a second period by usinq an estimation model based on the observation information observed in the second period;
acquiring difference information representing a difference between the first chronic stress value and the second chronic stress value and a difference between an event that occurred in the first period and an event that occurred in the second period; and
estimating a stressor relating to a chronic stress of the target person, based on the difference information.

11. A non-transitory computer readable storage medium storing a program executed by a computer, the program causing the computer to:
acquire observation information regardinq a target person, the observation information includinq a biological signal measured by a sensor worn by the target person;
estimate a first chronic stress value of the target person in a first by usinq an estimation model based on the observation information observed in the first period;
estimate a second chronic stress value of the target person in a second period by usinq an estimation model based on the observation information observed in the second period;
acquire difference information representing a difference between the first chronic stress value and the second chronic stress value and a difference between an event that occurred in the first period and an event that occurred in the second period; and
estimate a stressor relating to a chronic stress of the target person, based on the difference information.
